(19) United States
(12) Patent Application Publication
(10) Pub. No.: US 2023/0192605 A1
(43) Pub. Date: Jun. 22, 2023

(54) PROCESS FOR PRODUCTION OF INTERMEDIATES

(71) Applicant: DSM IP ASSETS B.V., HEERLEN (NL)

(72) Inventors: Werner BONRATH, Kaiseraugst (CH); Marc-André MUELLER, Kaiseraugst (CH); Bettina WUESTENBERG, Kaiseraugst (CH)

(21) Appl. No.: 17/915,998

(22) PCT Filed: Mar. 22, 2021
(86) PCT No.: PCT/EP2021/057241

§ 371 Date: Sep. 29, 2022

(30) Foreign Application Priority Data
Mar. 31, 2020 (EP) 20167022.1

(51) Int. Cl. C07C 403/02

(52) U.S. Cl. CPC C07C 403/02; C07C 2601/16

(57) ABSTRACT

The present invention relates to a new process for the production of specific intermediates, which are preferably used in the production of vitamin A and/or vitamin A acetate.

The present invention relates to a new process for the production of specific intermediates, which are preferably used in the production of vitamin A and/or vitamin A acetate.

Vitamin A

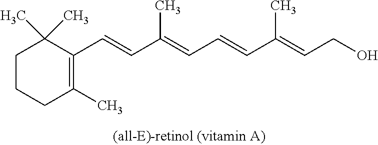 [SMILES: CC1=C(/C=C/C(C)=C/C=C/C(C)=C/CO)C(C)(C)CCC1]

or its derivatives such as Vitamin acetate is an important ingredient for many applications. Vitamin A plays a role in a variety of functions throughout the body, such as e.g. vision process, gene transcription, immune function, bone metabolism, haematopoiesis, skin and cellular health and antioxidant function.

Due to the importance of vitamin A (and its derivatives) and the complexity of the synthesis thereof, there is always a need for improved processes of production.

The goal of the present invention was to find easily accessible compounds, which can then be used in an improved synthesis of vitamin A or its derivates, preferably vitamin A (acetate). The aim was achieved by the synthesis as disclosed and described below.

The compound of formula (I)

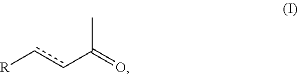

wherein R is one of the following formula

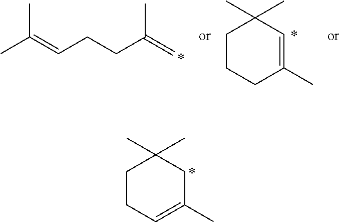

(* signifies where the moiety is attaching)

is reacted with a compound of formula (II)

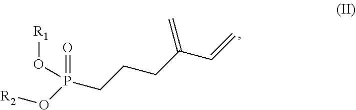

wherein

R₁ and R₂ are independently of each other C₁-C₄-alkyl.

The result of this reaction are compounds of formula (III)

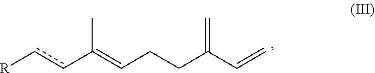

wherein R has the same meanings as defined above.

Therefore the present invention relates to the process (P) for the production of compounds of formula (III)

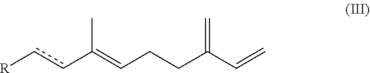

wherein R is one of the following formula

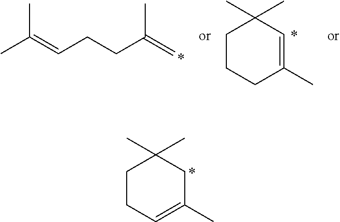

(* signifies where the moiety is attaching),

characterized in that a compound of formula (I)

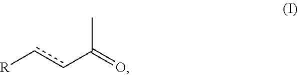

wherein R has the same definition as in formula (III) is reacted with a compound of formula (II)

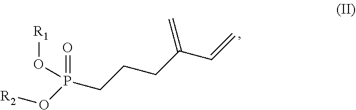

R₁ and R₂ are independently of each other C₁-C₄ alkyl.

The compounds of formula (III) as it can be seen from the formula can have an additional C—C double bond. This means the compound of formula (III) can be compound of formula (III′)

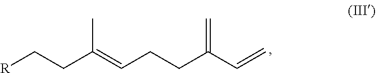

or the compound of formula (III″)

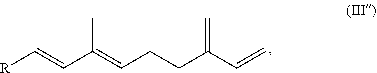

wherein R has the same definition as defined above.

It is obvious that also the compound of formula (I) can be either the compound of formula (I′) of the compound of formula (I″)

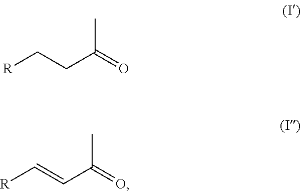

wherein R has the same definition as defined above.

Therefore the present invention relates to the process (P1), which is process (P), wherein the starting material is the compound of formula (I′)

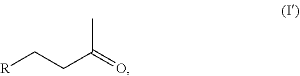

wherein R is one of the following formula

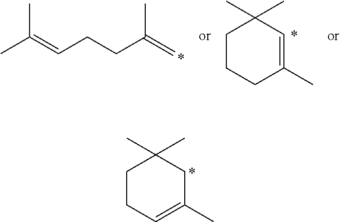

(* signifies where the moiety is attaching).

Therefore the present invention relates to the process (P2), which is process (P), wherein the starting material is the compound of formula (I″)

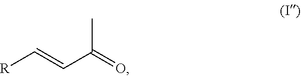

wherein R is one of the following formula

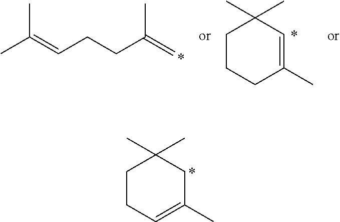

(* signifies where the moiety is attaching).

The compounds of formula (III) which are covered by the formula are the following:

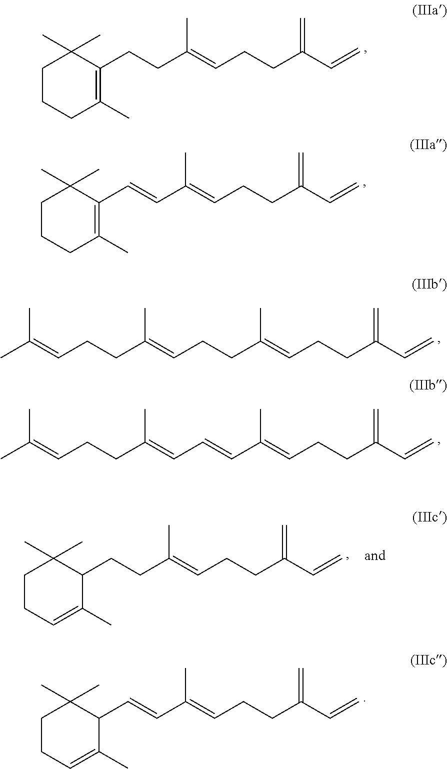

The starting material (compounds of formula (I) are the following ones:

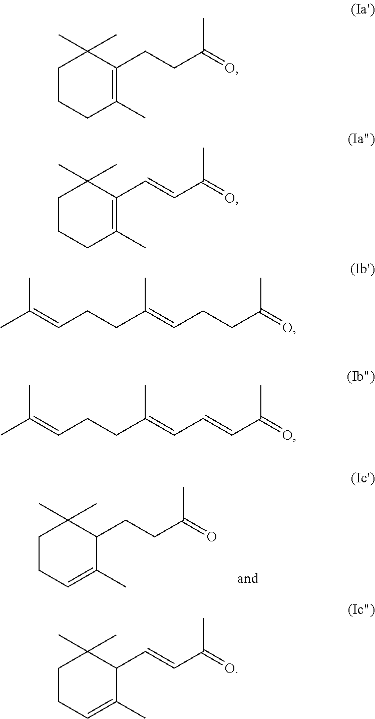

Therefore the present invention relates to the process (P1′), which is process (P) and (P1), wherein the starting material is the compound of formula (Ia′)

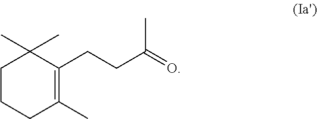

Therefore the present invention relates to the process (P1″), which is process (P) and (P1), wherein the starting material is the compound of formula (Ib′)

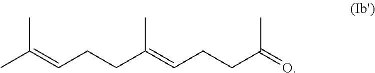

Therefore the present invention relates to the process (P1′″), which is process (P) and (P1), wherein the starting material is the compound of formula (Ic′)

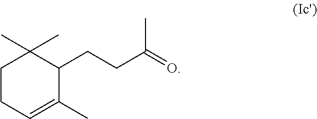

Therefore the present invention relates to the process (P2′), which is process (P) or (P2), wherein the starting material is the compound of formula (Ia″)

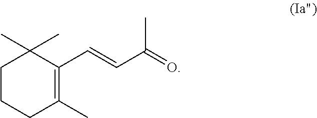

Therefore the present invention relates to the process (P2″), which is process (P) and (P1), wherein the starting material is the compound of formula (Ib″)

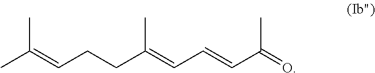

Therefore the present invention relates to the process (P2′″), which is process (P) and (P1), wherein the starting material is the compound of formula (Ic″)

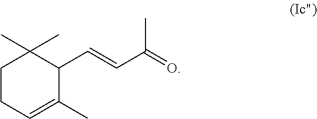

The process according to the present invention is usually carried out in the presence of a strong base such as Schlesinger base, 2,2,6,6-tetramethyl piperidine, lithium diisopropylamide, n-butyllithium, hexyllithium, tert.-butyl lithium, sec-butyllithium, metal amide (with metals such as Na, K and Cs), lithium hexamethyldisilazane, metal hydride (with metals such as Na, Mg, K and Cs), metal hydroxide (with metals such as Na, K and Cs), metal alkoxide (with metals such Na, K and Cs) or sodium hexamethyl-disilazane.

Therefore the present invention relates to the process (P3), which is process (P), (P1), (P1′), (P1″), (P1′″), (P2), (P2′), (P2″) or (P2′″), wherein the process is carried out in the presence of at least one strong base.

Therefore the present invention relates to the process (P3′), which is process (P3), wherein the at least one strong base is chosen from the group consisting of Schlesinger base, 2,2,6,6-tetramethyl piperidine, lithium diisopropylamide, n-butyllithium, hexyllithium, tert.-butyl lithium, sec-butyllithium, metal amide, lithium hexamethyldisilazane, metal hydride, metal hydroxide, metal alkoxide and sodium hexamethyl-disilazane.

Therefore the present invention relates to the process (P3″), which is process (P3), wherein the at least one strong base is chosen from the group consisting of Schlesinger base; 2,2,6,6-tetramethyl piperidine; lithium diisopropylamide; n-butyllithium; hexyllithium; tert.-butyl lithium; sec-butyllithium; metal amide, wherein the metal is chosen from the group consisting of Na, K and Cs; lithium hexamethyldisilazane; metal hydride, wherein the metal is chosen from the group consisting Na, Mg, K and Cs; metal hydroxide; wherein the metal is chosen from the group consisting Na, K and Cs, metal alkoxide; wherein the metal is chosen from the group consisting Na, K and Cs and sodium hexamethyl-disilazane.

The process is usually carried out in an inert solvent. Preferably the solvent is a polar aprotic solvent. More preferably the solvent is chosen from the group consisting of pyridine, toluene, xylene, THF, methyl THF, or ethers (such as diethylether, 1,4-dioxane, 1,2-dimethoxyethane and crown ethers).

Therefore the present invention relates to the process (P4), which is process (P), (P1), (P1′), (P1″), (P1′″), (P2), (P2′), (P2″), (P2′″), (P3), (P3′) or (P3″), wherein the process is carried out in at least one inert solvent.

Therefore the present invention relates to the process (P4′), which is process (P4), wherein the solvent is a polar aprotic solvent.

Therefore the present invention relates to the process (P4″), which is process (P4) or (P4′), wherein the at least one solvent is chosen from the group consisting of pyridine, toluene, xylene, THF, methyl THF, and ethers.

Therefore the present invention relates to the process (P4′″), which is process (P4) or (P4′), wherein the at least one solvent is chosen from the group consisting of pyridine, toluene, xylene, THF, methyl THF, and diethylether, 1,4-dioxane, 1,2-dimethoxyethane and crown ethers.

The process according to the present invention can be carried out at a temperature range of from −10° C. to 100° C., preferably at a temperature range of from −5° C. to 80° C., more preferably at a temperature range of from −5° C. to 30° C.

Therefore the present invention relates to the process (P5), which is process (P), (P1), (P1′), (P1″), (P1′″), (P2), (P2′), (P2″), (P2′″), (P3), (P3′), (P3″) (P4), (P4′), (P4″) or (P4′″), wherein the process is carried out at a temperature range of from −10° C. to 100° C.

Therefore the present invention relates to the process (P5′), which is process (P), (P1), (P1′), (P1″), (P1′″), (P2), (P2′), (P2″), (P2′″), (P3), (P3′), (P3″) (P4), (P4′), (P4″) or (P4′″), wherein the process is carried out at a temperature range of from 31 5° C. to 80° C.

Therefore the present invention relates to the process (P5″), which is process (P), (P1), (P1′), (P1″), (P1′″), (P2), (P2′), (P2″), (P2′″), (P3), (P3′), (P3″) (P4), (P4′), (P4″) or (P4′″), wherein the process is carried out at a temperature range of from −5° C. to 30° C.

The obtained products of the process according to the present invention (these are the compound of formula (III)) are ideal intermediates. Especially in the production of vitamin A and its derivates.

The following example serve to illustrate the invention. The temperature is given in ° C. and all percentages are related to the weight.

### EXAMPLES

### Example 1

### Synthesis of Compound of formula IIIa′

In a 10 ml two-necked flask, C₇-phosphonate (compound of formula (II)) (161 mg, 0.6 mmol) and dihydro-β-ionone (compound of formula (Ia′)) (108 mg, 0.5 mmol) were dissolved in anhydrous THF (3.0 ml). At 24° C., lithium diisopropylamide (0.50 ml, 2M in THF) was added dropwise within 5 min and stirred for 2 hours. Then GC measurement showed that the reaction was complete. Water (1 ml) was added carefully and the mixture was transferred into a separation funnel using 15 ml of dichloromethane and 15 ml of semi-saturated brine. The layers were separated, and the organic layer was washed with 15 ml of semi-saturated brine. The combined aqueous layers were extracted with 15 ml of dichloromethane. The combined organic layers were dried over sodium sulfate, filtered and evaporated to dryness under reduced pressure (rotavap) at 35° C. water-bath temperature. The crude product (compound of formula (IIIa′)) (231 mg, 34.45% purity by qNMR) was obtained in 58% yield and purified by column chromatography (SiO₂/heptane).

### Example 2

### Synthesis of Compound of Formula (IIIa″)

In a 10 ml two-necked flask, C₇-phosphonate (II) (161 mg, 0.6 mmol) and β-ionone (compound of formula Ia″)) (100 mg, 0.5 mmol) were dissolved in anhydrous THF (3.0 ml). At 24° C., lithium diisopropylamide (0.50 ml, 2M in THF) was added dropwise within 5 min and stirred for 2 hours. Then GC measurement showed that the reaction was complete. Water (1 ml) was added carefully and the mixture was transferred into a separation funnel using 15 ml of dichloromethane and 15 ml of semi-saturated brine. The layers were separated, and the organic layer was washed with 15 ml of semi-saturated brine. The combined aqueous layers were extracted with 15 ml of dichloromethane. The combined organic layers were dried over sodium sulfate, filtered and evaporated to dryness under reduced pressure (rotavap) at 35° C. water-bath temperature. After purification by column chromatography (SiO₂/heptane) 55.3 mg of product compound of formula (IIIa″) were obtained.

### Example 3

### Synthesis of Compound of Formula (IIIc″)

In a 10 ml two-necked flask, C₇-phosphonate (compound of formula (II)) (161 mg, 0.6 mmol) and α-ionone (compound of formula (Ic″)) (100 mg, 0.5 mmol) were dissolved in anhydrous THF (3.0 ml). At 24° C., lithium diisopropylamide (0.50 ml, 2M in THF) was added dropwise within 5 min and stirred for 2 hours. Then GC measurement showed that the reaction was complete. Water (1 ml) was added carefully and the mixture was transferred into a separation funnel using 15 ml of dichloromethane and 15 ml of semi-saturated brine. The layers were separated, and the organic layer was washed with 15 ml of semi-saturated brine. The combined aqueous layers were extracted with 15 ml of dichloromethane. The combined organic layers were dried over sodium sulfate, filtered and evaporated to dryness under reduced pressure (rotavap) at 35° C. water-bath temperature. After purification by column chromatography (SiO₂/heptane) the product compound of formula (IIIc″) was obtained in 36% yield (48.7 mg).

1. Process for the production of compounds of formula (III) 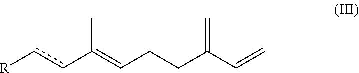
wherein R is one of the following formula 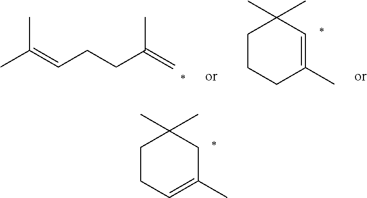
(* signifies where the moiety is attaching),
characterized in that a compound of formula (I) 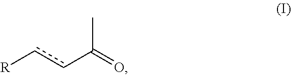
wherein R has the same definition as in formula (III) is reacted with a compound of formula (II) 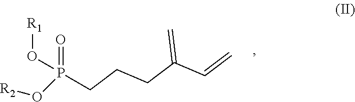
R₁ and R₂ are independently of each other C₁-C₄ alkyl.

2. Process according to claim 1, wherein the starting material is the compound of formula (I′) 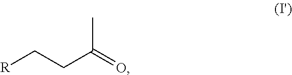
wherein R is one of the following formula 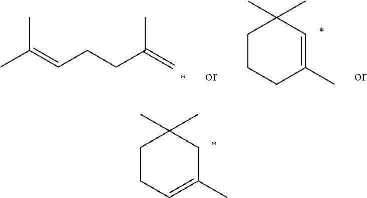
(*signifies where the moiety is attaching).

3. Process according to claim 1, wherein the starting material is the compound of formula (I″) 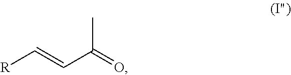
wherein R is one of the following formula 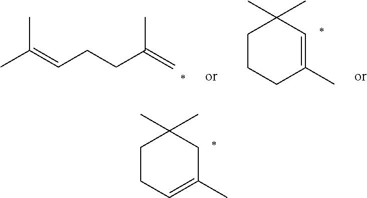
(* signifies where the moiety is attaching).

4. Process according to claim 1, wherein the process is carried out in the presence of at least one strong base.

5. Process according to claim 4, wherein the at least one strong base is chosen from the group consisting of Schlesinger base, 2,2,6,6-tetramethyl piperidine, lithium diisopropylamide, n-butyllithium, hexyllithium, tert.-butyl lithium, sec-butyllithium, metal amide, lithium hexamethyldisilazane, metal hydride, metal hydroxide, metal alkoxide and sodium hexamethyl-disilazane.

6. Process according to claim 1, wherein the process is carried out in at least one inert solvent.

7. Process according to claim 6, wherein the solvent is a polar aprotic solvent.

8. Process according to claim 6, wherein the at least one solvent is chosen from the group consisting of of pyridine, toluene, xylene, THF, methyl THF, and ethers.

9. Process according to claim 1, wherein the process is carried out at a temperature range of from −10° C. to 100° C.
